(19) United States
(12) Patent Application Publication
(10) Pub. No.: US 2023/0272338 A1
(43) Pub. Date: Aug. 31, 2023

(54) PROCESS FOR PRODUCING CARRIER PARTICLES FOR THE CULTIVATION OF BIOLOGICAL CELLS, CARRIER PARTICLES AND THEIR USE

(71) Applicant: Fraunhofer-Gesellschaft zur Foerderung der angewandten Forschung e. V., Muenchen (DE)

(72) Inventors: Heiko ZIMMERMANN, Sulzbach (DE); Julia NEUBAUER, Wuerzburg (DE); Michael GEPP, Wuerzburg (DE)

(21) Appl. No.: 18/009,071

(22) PCT Filed: Jun. 15, 2021
(86) PCT No.: PCT/EP2021/066071

§ 371 Date: Dec. 8, 2022

(30) Foreign Application Priority Data
Jun. 18, 2020 (DE) 102 020 116 108.5

(51) Int. Cl. C12N 5/00

(52) U.S. Cl. CPC C12N 5/0075; C12N 2531/00; C12N 2533/20; C12N 2533/74; C12N 2537/10

(57) ABSTRACT

The invention relates to a process for producing carrier particles for the cultivation of biological cells, comprising the steps of providing an aqueous suspension of hydrogel beads and freeze-drying of the hydrogel beads so that dry hydrogel particles are formed, wherein at least one lyoprotectant substance is added to the suspension, the hydrogel beads are loaded in the suspension with the lyoprotectant substance and the dry hydrogel particles receive a shape under the effect of the lyoprotectant substance, which shape approximates a spherical particle shape and is still maintained after rehydrating. The invention also relates to carrier particles for a cultivation of biological cells, which comprise dry hydrogel particles, preferably having a protein coating, and to applications of the carrier particles.

The invention relates to a method for producing carrier particles for culturing biological cells, in particular a method for producing dried hydrogel particles. The invention further relates to carrier particles for culturing biological cells, in particular dried hydrogel particles, which have been produced using said method, and to applications of the carrier particles. The invention is applicable to the culture of biological cells, in particular in the culture (expansion) of multipotent and pluripotent stem cells or of differentiated cells, e.g. for tissue engineering.

The present description will make reference to the following prior art, which represents the technical background of the invention:
Dissertation “Investigation of structural cellular processes in the context of tissue engineering: alginate-based scaffold structures” Michael Gepp, Universität des Saarlandes, 2017;
EP 2 361 968 A1;
US 6 642 363 B1; and
M. Gepp et al. in “J. Appl. Phycol.” (2017) 29:2451-2461.

The use of polysaccharide polymers, in particular of alginate hydrogels (also abbreviated to “alginates”), as carriers for cell cultures is generally known (see [1] and the literature cited therein). Alginates can be used in layered form on two-dimensional substrates, or as cell carriers (spherical particles, beads, carrier beads, microcarriers) in suspensions (see e.g. [1], [2]). Depending on the specific culturing task and the cells to be cultured, alginates can be modified with additives. Additives comprise e.g. peptides or collagen, which influence cell adhesion (see e.g. [3] or [4]). Culturing cells on microcarriers in suspension, e.g. in bioreactors, has advantages for the formation of three-dimensional arrangements of cells (e.g. 3D aggregates, spheroids, microcarrier-cell hybrids) under close-to-physiological conditions, and it enables efficient performance of processes by making it possible to set a favorable surface-to-volume ratio.

Conventional cell culture using alginates as microcarriers in a bioreactor comprises for example the following steps. Firstly, alginate beads are produced by precipitating liquid alginate in spherical form by crosslinking (see e.g. [1]). A suspension of alginate beads is formed which, depending on the application, have a size distribution with diameters in the range from 200 µm bis 500 µm. The width of the size distribution, which can be set on the basis of the conditions of droplet formation, can have an effect on the subsequent cell culture. After washing the crosslinked alginate beads, they are modified (activated and/or functionalized) e.g. by coupling tyramine or adjusting the elasticity of the beads by means of alginate mixtures selected before the alginate beads are produced. The finished modified alginate beads are suspended in a culture medium and placed in a bioreactor. The cells to be cultured are added to the suspension in the bioreactor, where they colonize the alginate beads and are subjected to a specified culture protocol.

The conventional method has the following disadvantages and limitations. Producing the modified alginate beads with predefined properties requires expert knowledge, making it difficult to routinely provide the alginate beads in a laboratory for cell culture or for industrial use. The alginate beads are typically produced according to the user’s specifications, e.g. with a specific size distribution, diameter and/or modification, delivered to the user in suspension and initially stored at the user’s site. However, one disadvantage is that the transport and storage of the suspension at the user’s site may undesirably alter the alginate beads.

Alginate beads can aggregate (clump together) or disintegrate, which is disadvantageous for precise and reproducible metering by the user. The sterility of the alginate beads can be lost on storage. Furthermore, properties of the finished alginate beads can only be checked or varied by the user to a highly limited extent, or not at all. For example, the size distribution of alginate beads in a suspension can only be subsequently changed, if at all, with a great deal of effort. The water content of the alginate beads can distort the culture medium in the bioreactor. To prevent this, the number of alginate beads can be kept small, but this leads to high consumption of medium and a low number of culturable cells. Therefore, conventional methods for cell culture are characterized by low efficiency, high process costs, lack of scalability, low success rate and low reproducibility.

It is also known to subject alginate beads to drying ([1]). Dried alginate beads can be stored better than those in suspension. Furthermore, dried alginate beads can be rehydrated with the culture medium in the bioreactor. However, it has not yet been possible to overcome the limitations of the conventional uses of alginate beads in practice. Even when using conventionally dried and rehydrated alginate beads, the dried alginate beads have proven difficult to handle and the reliability of successful cell culturing, and the reproducibility of the culturing result, are limited even when using unchanging culture protocols.

It is the objective of the invention to provide an improved method for producing carrier particles for culturing biological cells, in particular an improved method for producing dried hydrogel particles, which makes it possible to overcome the disadvantages of conventional techniques. It is also the objective of the invention to provide improved carrier particles for culturing biological cells, which make it possible to overcome the disadvantages of conventional carrier particles . The invention is intended in particular to provide carrier particles which have reproducible properties, improved storability, can be precisely metered, are easy to modify and/or are suitable for routine applications in bioreactors of different constructions and purposes. Carrier particles produced according to the invention are in particular intended to enable cell culturing with increased efficiency, success rate and/or reproducibility.

These objectives are each achieved by a method for producing carrier particles for culturing biological cells and by carrier particles having the features of the independent claims. Preferred embodiments and applications of the invention are given in the dependent claims.

According to a first general aspect of the invention, the above objective is achieved by a method for producing carrier particles for culturing biological cells, which method comprises the following steps. An aqueous suspension of spherical hydrogel beads is provided. Preferably, the hydrogel beads are freshly produced by crosslinking a precursor polymer with an ionic precipitant. The hydrogel beads can be produced using methods which are known per se. The suspension liquid of the aqueous suspension of hydrogel beads comprises an aqueous solution with the precipitant in which the hydrogel beads are produced, and/or a washing and/or buffer solution by means of which the hydrogel beads are optionally washed after the precipitation. The hydrogel beads are then freeze-dried, so that dried hydrogel particles are formed. The freeze-drying of the hydrogel beads comprises applying a reduced temperature (temperature below room temperature, preferably below 0° C.) and a negative pressure (pressure lower than atmospheric pressure) to the hydrogel beads. The freeze-drying of the hydrogel beads preferably takes place in the suspended state with the suspension liquid, with the suspension liquid first being removed and the hydrogel beads then being dried. Alternatively, the hydrogel beads can be removed from the suspension liquid of the aqueous suspension before the freeze-drying, i.e. the suspension liquid is separated off from the hydrogel beads before the freeze-drying.

According to the invention, at least one lyoprotectant substance is added to the suspension liquid, the hydrogel beads in the suspension being loaded with the lyoprotectant substance before the freeze-drying. The lyoprotectant substance (or lyoprotectant) is a substance which minimizes or prevents damage to the hydrogel beads, in particular to the macromolecules from which the hydrogel beads are constructed, by ice formation during the freeze-drying. In addition, a lyoprotectant substance is used which causes the hydrogel beads after the freeze-drying, i.e. the dried hydrogel particles, to have a shape approximated to a spherical particle shape under the action of the lyoprotectant substance.

The hydrogel beads preferably comprise alginate beads (alginate hydrogels). However, the implementation of the invention is not limited to alginate beads in practice, but rather can also accordingly be implemented with collagen, gellan or pectin hydrogels. Alginate has proven particularly advantageous because it has a shape-memory effect and re-forms spherical alginate beads after the freeze-drying and rehydration.

The term “approximately spherical particle shape” includes a shape of a dried hydrogel particle which represents a spheroid (in particular sphere or ellipsoid) and has a smooth or structured surface topology with characteristic structural dimensions, for example steps, protrusions or depressions, which are smaller than a cross-sectional dimension, preferably less than ⅒ of the cross-sectional dimension, of the dried hydrogel particle.

According to a second general aspect of the invention, the objective is achieved by carrier particles for culturing biological cells, which carrier particles comprise dried hydrogel particles which contain a lyoprotectant substance and have an approximately spherical shape. The carrier particles are preferably produced using the stated method according to the first general aspect of the invention or one of the embodiments thereof. Preferably, the carrier particles have a characteristic cross-sectional dimension, for example a diameter, in the range from 50 µm to 2 mm.

According to a third general aspect of the invention, the above objective is achieved by uses of the carrier particles according to the second general aspect of the invention or the embodiments thereof as a cell carrier for culturing biological cells. According to preferred variants, the use of the carrier particles in a suspension bioreactor, in particular a culture vessel, a microtitration plate, suspended droplets, a cell culture bag, a (suspension) bioreactor and/or a dish, for example a Petri dish, can be provided. The use of the carrier particles preferably comprises the phases of producing the dried particles, rehydration and washing, cell inoculation, cell propagation and cell passage/harvesting.

The inventors have found that by loading, according to the invention, the hydrogel beads with the lyoprotectant substance, which causes the approximately spherical particle shape of the dried hydrogel particles, the disadvantages of conventional suspended or dried alginate beads in terms of storability, meterability and handleability are avoided. A gentle freeze-drying, retaining the structure and function of the spherical hydrogel beads, is achieved.

Storability is improved, since adhesion or aggregation is prevented, sterility can be better maintained in the dry state, and thus contamination of the prepared carrier beads can be prevented. These advantages also apply in particular with respect to the conventional approaches to freeze-drying of alginate beads, e.g. according to [1]. Thus, the inventors have found that the conventional alginate beads in the dried state do not have a spherical shape but rather an irregularly deformed shape, e.g. folded and/or provided with cracks or protrusions, which impairs the quality of the dried alginate beads, e.g. due to density fluctuations in the bulk material and irregular clump formation. Dried hydrogel particles produced according to the invention promote sterile lyophilization and rehydration.

Meterability is improved, since the dried hydrogel particles form a homogeneous material which is entirely flowable or forms a cake which can be cut up (lyocake, also referred to as dried product matrix). The accuracy and reproducibility in setting the concentration of the carrier beads in the reactor is improved. The dried hydrogel particles can be quantified e.g. by a volume measurement, a weight measurement and/or an optical measurement. Since the dried hydrogel particles are opaque, the rehydration in the bioreactor can be verified with the optical measurement.

The dried hydrogel particles can be processed in a simplified manner. The processing is preferably carried out in a sterile cabinet, but pipetting is not required for the metering. The dried hydrogel particles can be pre-portioned in a simplified manner. Improved quality management is enabled in the process of producing and using the dried hydrogel particles.

The dried hydrogel particles can be used as a pourable bulk material or in pellet form, and can in particular be introduced directly into a bioreactor (vessel in which the cell culturing takes place and which enables setting of biochemical and physical culture conditions). Advantageously, the dried hydrogel particles do not distort the overall volume of the culture medium in the bioreactor.

The rehydrated hydrogel beads are preferably used by the dried hydrogel particles being directly added to the culture medium in the bioreactor. The cells to be cultured may already be suspended in the culture medium when the dried hydrogel particles are fed in, or they may be added after the rehydration of the dried hydrogel particles in the bioreactor. The degree of rehydration of the dried hydrogel particles is preferably determined using an optical transmission measurement or scattered light measurement on the culture medium. The cells are particularly preferably fed into the culture medium after the dried hydrogel particles have been completely hydrated.

The rehydration of the dried hydrogel particles, e.g. in water, can optionally be considered a further sub-step of the method according to the invention. Advantageously, the hydrogel beads formed upon rehydration (rehydrated hydrogel beads) have a spherical particle shape also after the rehydration. Preferably, the rehydrated hydrogel beads are characterized by the spherical shape that they had before the freeze-drying. This promotes the culturing of cells on the hydrogel beads in an inoculation phase. In addition, spherical rehydrated hydrogel beads are advantageously characterized by favorable floating behavior in the suspension and a reduced tendency towards undesired aggregations compared to deformed hydrogel beads.

According to a preferred embodiment of the invention, the hydrogels are loaded with a protein layer. This modification is preferably carried out after producing the hydrogel beads and before the freeze-drying. The protein layer may comprise e.g. extracellular matrix proteins, thereby advantageously promoting adhesion of the cells to rehydrated hydrogel beads. The protein layer may comprise a submonolayer, a monolayer or a multilayer of protein molecules on the hydrogel beads. For example, after producing the hydrogel beads in a suspension, the at least one protein to be coupled is added to the suspension liquid and incubated with the hydrogel beads. Advantageously, the incubation enables covalent coupling of at least one protein or a mediator molecule to the whole surface, including any pores, of the hydrogel beads.

The loading with the protein layer preferably takes place before the freeze-drying. In selected applications of the invention, the loading with the protein layer may take place after the freeze-drying, in particular after the rehydration, e.g. in the bioreactor. For example, at least one protein may be supplied from the culture medium in the bioreactor or from cells already present in the bioreactor.

The hydrogel beads are preferably loaded with at least one mediator molecule (e.g. tyramine) and/or at least one of the proteins comprising laminin (recombinant and tissue-specific), vitronectin (recombinant and compatible with pluripotent stem cells), collagen (tissue-specific, compatible with multipotent stem cells), proteins from decellularized tissue (highly tissue-specific) and complex protein mixtures from the basal matrix (e.g. Geltrex, Matrigel, denovoMatrix).

According to a further preferred embodiment of the invention, residues of the lyoprotectant substance are removed after the freeze-drying. Separating off residues of the lyoprotectant substance from the dried hydrogel particles results in advantages for the later rehydration and subsequent cell culturing. Advantageously, influences of the lyoprotectant substance on the cells are prevented or minimized. The removal of residues of the lyoprotectant substance comprises complete elimination of the lyoprotectant substance from the dried hydrogel particles or elimination of the lyoprotectant substance from the surfaces of the dried hydrogel particles. The surfaces of the dried hydrogel particles are particularly preferably free of residues of the lyoprotectant substance.

The residues of the lyoprotectant substance, for example the “lyocake”, are particularly preferably removed by mechanical processing of the dried material, comprising disintegration thereof and screening. Advantageously, the dried hydrogel particles are separated from one another at the same time as the lyoprotectant substance is separated from the dried hydrogel.

Alternatively or additionally, according to a further variant of the invention, residues of the dried lyoprotectant substance can be removed after the rehydration of the dried hydrogel particles and before using the particles in a washing solution, in particular composed of sodium chloride. By washing the rehydrated hydrogel beads, an influence of the lyoprotectant substance on the subsequent cell culturing is advantageously further suppressed.

A further advantage of the invention consists in being different lyoprotectant substances available for the modification of the hydrogel beads according to the invention. The lyoprotectant substance may particularly comprise trehalose, dimethyl sulfoxide (DMSO), sucrose and/or a poloxamer. These lyoprotectants, which can be used individually or in combination, have the advantage that their effect on biological cells has been thoroughly investigated. Undesired effects on the cell culture can therefore be avoided or minimized. Trehalose and/or sucrose are particularly preferably used as lyoprotectant substance, because when they are used, carrier particles are obtained which, after rehydration, are characterized by particularly comprehensive shape and function retention and by particularly good cell adhesion in the cell culture.

According to a further preferred embodiment of the invention, the concentration of the lyoprotectant substance in the suspension liquid with the originally produced hydrogel beads is selected in the range from 1 mg/ml to 500 mg/ml. This concentration range is preferred because, below the concentration of 1 mg/ml, the spherical particle shape is only insufficiently approximated and, above 500 mg/ml, an excessive load on the culture medium in the bioreactor can arise because of the lyoprotectant substance.

According to a further advantageous embodiment of the invention, functionalization of the cell carriers produced according to the invention for culturing biological cells can be provided. Preferably, functionalization of the hydrogel beads takes place before or during production thereof (encapsulation of active ingredients, magnetic particles, etc.), e.g. before or during dripping for producing the hydrogel beads by precipitation, in order for the substances to be enclosed in the hydrogel matrix, or after provision thereof and before freeze-drying (e.g. surface functionalization with tyramine and/or proteins). The functionalization comprises the addition of particles and/or substances which afford the hydrogel beads further properties which go beyond the carrier function. According to preferred variants of the invention, the hydrogel beads are loaded with magnetic particles and/or biologically active substances (active ingredients). The magnetic particles and/or active ingredients are particularly preferably supplied before the freeze-drying. The inventors have determined that the functionalized hydrogel beads in the dried state also form particles with an approximately spherical shape and in the rehydrated state also form spherical cell carriers.

Hydrogel beads which each contain one or more magnetic particles advantageously offer the possibility of manipulating the cell carriers in the bioreactor by means of magnetic fields. The magnetic particles may consist of permanent magnet materials which are known per se.

As biologically active substances for modifying hydrogel beads, use is preferably made of differentiation factors, i.e. biologically active substances which trigger cell differentiation and/or influence the direction of the cell differentiation at a specific point in time.

If, according to a further preferred embodiment of the invention, the dispersion of the originally provided hydrogel beads contains a cohesion-reducing substance, adhesion of the dried hydrogel particles to one another is suppressed. This advantageously promotes pourability of the dried hydrogel particles. The cohesion-reducing substance particularly preferably comprises polyethylene glycol, the effect of which on biological cells has advantageously been thoroughly investigated.

For the freeze-drying of the hydrogel beads, a freeze-drying method known per se can be selected. For the freeze-drying, in particular of alginate beads, a protocol having the following phases is preferably applied. Firstly, there is a freezing phase, in which the hydrogel beads provided in suspension are frozen according to a predefined time-temperature function with a freezing interval of at least 120 min and an end temperature below -20° C. and above -80° C. Preferably, freezing rates in the range from 50° C./min (rapid freezing), through 1 to 1.5° C./min (moderate freezing), to 0.1 to 1° C./min (slow freezing) are set. The setting in the time-temperature function advantageously enables gentle freezing of the hydrogel beads. Subsequently, a stabilization phase is provided, in which the hydrogel beads are stored at the end temperature for a stabilization interval duration of at least 90 min. Thereafter, a first negative pressure selected in the range from 30 µbar to 60 µbar is applied to the frozen hydrogel beads at the end temperature in a first drying phase for forming the dried hydrogel particles. A second drying phase follows, in which a second negative pressure, which is lower than the first negative pressure, is applied to the dry hydrogel particles at a temperature equal to or greater than the end temperature. Finally, a ventilation phase follows, in which the dried hydrogel particles are transferred to a normal pressure according to a time-pressure function having a pressure increase interval of at least 0.5 min to 1 min (1 bar/min). The ventilation phase takes place in a vessel with application of an inert gas or air.

Further details and advantages of the invention are described below with reference to the appended drawings. The drawings show:

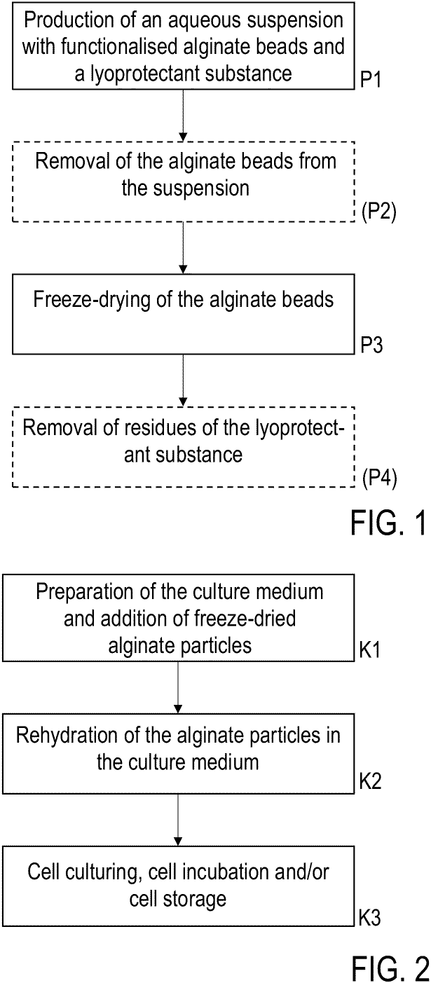

FIG. 1: a flow chart showing features of preferred embodiments of the method according to the invention for producing carrier particles;

FIG. 2: a flow chart showing features of preferred embodiments of the use of carrier particles produced according to the invention;

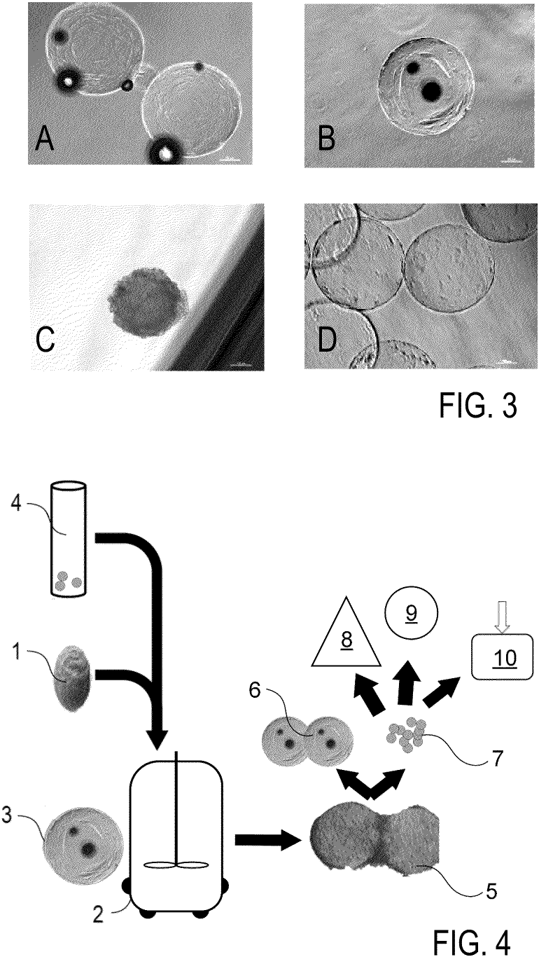

FIG. 3: photographs of rehydrated carrier particles compared to conventional carrier particles; and

FIG. 4: a schematic depiction of the method for an exemplary application of carrier particles produced according to the invention.

The invention is described below with exemplary reference to alginate-based cell carriers (carrier particles). Alginate beads, which have been loaded according to the invention with the lyoprotectant substance and subjected to freeze-drying, can for example be produced from commercially available alginate, which typically has a low viscosity due to relatively short chain lengths of the polymer macromolecules. Alternatively, use may be made of alginate having a higher viscosity, due to longer molecule chains, than the commercially available alginate. The selection of a specific alginate to be used is preferably made on the basis of the desired elasticity of the cell carrier during the cell culturing. The invention is not restricted to the use of alginate, but rather can also accordingly be carried out with other hydrogels, for example collagen, gellan or pectin. Embodiments of the invention are described below in particular with reference to examples for loading alginate with lyoprotectant substances, modifying alginate beads, dried alginate particles and/or rehydrated alginate beads, and the protocols for freeze-drying. Details of the use of the rehydrated alginate beads in the culturing of biological cells can be carried out as is known per se from conventional cell culturing.

FIG. 1 schematically shows the main steps of the production, according to the invention, of dried alginate particles. In step P1, alginate beads are produced in an aqueous suspension in a container. The alginate beads are produced for example by generating Na-alginate droplets using a nozzle and crosslinking the alginate droplets in an aqueous suspension liquid with an ionic precipitant, for example as described in [1]. A BaCl₂ solution, for example, is used as precipitant. The size and size distribution of the alginate beads can be set by the dimension of the nozzle and the operating parameters of the nozzle. The crosslinked alginate beads form dimensionally stable alginate beads which are suspended in the suspension liquid.

The alginate beads are preferably functionalized after the precipitation by coating with tyramine and/or a protein, for example Matrigel. The coating takes place by sedimentation from the suspension liquid, or direct covalent coupling. The Matrigel coating affords advantages for the adhesion of cells in a subsequent cell culturing.

A lyoprotectant substance is already added to the suspension liquid while the alginate droplets are being dropped into the suspension liquid, or alternatively after the crosslinking and formation of the alginate beads. In the inventor’s tests, trehalose (100 mg/ml), poloxamer (trade name Pluronic F 68, 1 mg/ml), and/or sucrose (100 mg/ml) in an aqueous NaCl solution (0.9%), for example, were used as lyoprotectant substance. The suspended alginate beads are loaded with the lyoprotectant substance for example by storing the alginate beads in an aqueous solution containing the lyoprotectant substance, e.g. for at least one day.

Optionally, in step P2, the alginate beads loaded with the lyoprotectant substance are removed from the suspension. For example, the suspension liquid is decanted, leaving the alginate beads surrounded by residual liquid in the container. Alternatively, a screen is used for the removal.

If step P2 is not provided, in step P3 the freeze-drying of the alginate beads takes place immediately after the alginate beads are loaded with the lyoprotectant substance. The following phases are provided for this purpose.

Firstly, the alginate beads are cooled to an end temperature of e.g. -45° C. in a freezing phase over 150 min (approx. 0.4° C./min). During the freezing phase, a linear time-temperature function is for example applied.

Subsequently, a stabilization phase follows, in which the alginate beads are stored at the end temperature for a stabilization interval duration of 120 min. The stabilization phase has the advantage that the sample is completely frozen through with the frozen alginate beads before the drying phases are carried out.

A subsequent first drying phase has the function of a drying up phase, in which the suspension liquid is removed by means of sublimation. The first drying phase is performed for example in a lyophilizer with a cooling unit and a condenser. During the first drying phase, the end temperature, for example -45° C., is maintained, while the pressure is lowered following a linear time-pressure function over a duration of 10 min from atmospheric pressure down to a first negative pressure of 50 µbar. Subsequently, in the first drying phase, the frozen sample is held at the end temperature and the first negative pressure for a stabilization duration of for example 80 hours.

In a subsequent second drying phase, a second negative pressure, which is lower than the first negative pressure and is for example 100 µbar, is applied to the dried alginate particles. The lowering to the second negative pressure takes place with a linear time-pressure function over a duration of for example 300 min. During the second drying phase, the temperature of the dried alginate particles is equal to the end temperature or an increased temperature, for example room temperature (20° C.). After lowering to the second negative pressure, the dried sample is kept at the second negative pressure during the second drying phase for a stabilization duration of for example 20 hours.

Subsequently, a ventilation phase is provided, in which the dried alginate particles are transferred to a normal pressure according to a linear time-pressure function having a pressure increase interval of at least 1 min. The ventilation phase can be provided using air or an inert gas. Applying a relatively long pressure increase interval advantageously prevents damage to the dried alginate particles. If ventilating with air, the storage vessel is closed beforehand in the chamber, whereas when using an inert gas, the closure takes place after the ventilation.

Dry nitrogen or argon is preferably used as inert gas. The dried alginate particles are particularly preferably stored in the inert gas or in a vacuum. Practical tests gave a storability, e.g. at 4° C., of the dried alginate particles produced according to the invention without any loss of functionality for several months.

The method for freeze-drying P3 described by way of example can be modified, in terms of the temperatures and pressures set and the form of the time-pressure and time-temperature functions, on the basis of the specific conditions of application. For example, preparatory tests can be used to determine what time and pressure parameters deliver optimal drying results for a specific hydrogel, in particular alginate, sample.

As a result of the freeze-drying P3, the dried alginate particles are present as finished cell carriers. Because of the addition, according to the invention, of the lyoprotectant substance, the dried alginate particles have a spherical shape (see for example the photograph in FIG. 4), which advantageously also remains after rehydration. The dried particles can be joined together as a cake or form a pourable bulk material composed of individual particles. The formation of the pourable bulk material is promoted if a cohesion-reducing substance, for example polyethylene glycol, is added to the suspension of the alginate beads in addition to the lyoprotectant substance. Use is for example made of polyethylene glycol having the trade name PEG 600 at a concentration of 50 mg/ml in order to minimize adhesion of the dried alginate particles to one another.

Depending on the concentration of the lyoprotectant substance used, said substance may form residues on the surface of the dried alginate particles following the freeze-drying P3. Accordingly, as shown in FIG. 1, an optional step P4 may be provided for removing the lyoprotectant substance, in particular from the surfaces of the dried alginate particles, for example by screening or grinding.

An application of the dried alginate particles as cell carriers in the culturing of biological cells is shown schematically in FIG. 2. Firstly, in step K1, an aqueous culture medium is prepared in a bioreactor. The composition of the culture medium is selected on the basis of the specific application of the cell culture in a manner known per se. The alginate particles produced according to the invention are added to the culture medium. The dried alginate particles are metered in for example by means of weighing. Tests with dried alginate particles produced according to the invention showed that particles without polyethylene glycol loading initially lay on the surface of the culture medium and only sank into the culture medium after centrifugation, whereas with particles modified with polyethylene glycol, no floating on the culture medium was observed.

In the aqueous culture medium, in step K2, the rehydration of the alginate particles takes place. The dried alginate particles are converted into alginate beads by absorbing water from the culture medium, as shown for example in FIG. 3. Subsequently, in step K3, the cell culturing, cell incubation and/or cell storage of biological cells takes place in the culture medium with the alginate beads.

FIG. 3 shows light-microscopic images of alginate beads which, according to preferred variants of the invention, have been loaded with trehalose (A) or sucrose (B), freeze-dried and rehydrated, in comparison with alginate beads which have been freeze-dried and rehydrated without a lyoprotectant substance (C), and freshly produced alginate beads (D) in suspension (diameter approx. 400 µm). The rehydrated particles (A, B) produced according to the invention advantageously clearly exhibit the same spherical shape as the suspended, untreated alginate beads (D). On the contrary, the untreated freeze-dried and rehydrated particles (C) (e.g. according to [1]) have a bumpy and irregular surface unlike the untreated alginate beads.

A further application of the dried alginate particles as cell carriers in the cell expansion of hiPS cells (human induced pluripotent stem cells) is schematically shown in FIG. 4. For a first expansion phase, dried alginate particles 1 are added to a first culture vessel 2, e.g. a suspension bioreactor having a volume of a few milliliters (e.g. 10 ml) to a few liters (e.g. 3 I). Alginate beads 3 (shown by way of example) are formed by rehydrating from the alginate particles 1. In addition, hiPSC cells are added to the culture vessel 2 from a vessel 4. The cell culturing then takes place under specified culture conditions, with the cells initially attaching and adhering to the microcarriers and then multiplying by a factor (e.g. sevenfold) over several days (e.g. 7 days). In a final step of the process, the alginate beads with adhered cells 5 are obtained; i.e., after they are obtained (e.g. by enzymatic treatment), the consumed microcarriers 6 and the multiplied hiPS cells 7 are present. These can subsequently be stored in a cell bank 10, further investigated and/or processed 9 (e.g. differentiation into cardiomyocytes) and/or transferred for direct use 8 (e.g. bioprinting).

The features of the invention disclosed in the above description and in the drawings and the claims can be significant, both individually and in combination or sub-combination, for carrying out the invention in the various configurations thereof.

1. A method for producing carrier particles for culturing biological cells, comprising the steps of:
providing an aqueous suspension of hydrogel beads, and
freeze-drying the hydrogel beads such that dried hydrogel particles are formed, wherein
at least one lyoprotectant substance is added to the aqueous suspension, wherein the hydrogel beads in the aqueous suspension are loaded with the at least one lyoprotectant substance and, under the effect of the at least one lyoprotectant substance, the dried hydrogel particles obtain a shape approximated to a spherical particle shape.

2. The method according to claim 1, wherein
the hydrogel beads are loaded with a protein layer.

3. The method according to claim 1, wherein
residues of the at least one lyoprotectant substance are removed after the freeze-drying.

4. The method according to claim 3, wherein
the residues are removed by mechanical processing of the dried particles, comprising disintegration and screening.

5. The method according to claim 1, wherein
the at least one lyoprotectant substance comprises at least one of trehalose, dimethyl sulfoxide, sucrose and a poloxamer.

6. The method according to claim 1, wherein
a concentration of the at least one lyoprotectant substance in the aqueous suspension is selected to be in a range from 1 mg/ml to 500 mg/ml.

7. The method according to claim 1, wherein
the dried hydrogel particles are subjected to rehydration, and
residues of dried lyoprotectant substance are removed before using the carrier particles in a washing solution comprising sodium chloride.

8. The method according to claim 1, wherein
the hydrogel beads contain at least one of magnetic particles and biologically active substances, wherein the at least one of the magnetic particles and the biologically active substances are dried with the hydrogel beads during the freeze-drying.

9. The method according to claim 8, wherein
the hydrogel beads contain differentiation factors.

10. The method according to claim 1, wherein
at least one cohesion-reducing substance, which promotes pourability of the dried hydrogel particles, is added to the aqueous supension.

11. The method according to claim 10, wherein
the at least one cohesion-reducing substance comprises polyethylene glycol.

12. The method according to claim 1, wherein the freeze-drying of the hydrogel beads comprises the following phases:
a freezing phase, in which the hydrogel beads are frozen according to a time-temperature function with a freezing interval of at least 120 min and an end temperature of less than -40° C.,
a stabilization phase, in which the hydrogel beads are stored at the end temperature for a stabilization interval duration of at least 90 min,
a first drying phase for forming the dried hydrogel particles, in which a first negative pressure, which is selected in a range from 30 µbar to 60 µbar, is applied to the frozen hydrogel beads at the end temperature,
a second drying phase, in which a second negative pressure, which is lower than the first negative pressure, is applied to the dried hydrogel particles at a temperature equal to or greater than the end temperature, and
a ventilation phase, in which the dried hydrogel particles are transferred to a normal pressure according to a time-pressure function having a pressure increase interval of at least 0.5 min.

13. Carrier particles for culturing biological cells, comprising
dried hydrogel particles having a protein coating, wherein the dried hydrogel particles contain a lyoprotectant substance and have a shape approximated to a spherical shape.

14. The carrier particles according to claim 13, wherein
the protein coating comprises at least one of Matrigel, collagen, laminin and vitronectin.

15. The carrier particles according to claim 13, wherein
surfaces of the carrier particles are free of residues of the lyoprotectant substance.

16. The carrier particles according to claim 13,
having a characteristic cross-sectional dimension in a range from 50 µm to 2 mm.

17. The carrier particles according to claim 13, containing at least one of
magnetic particles, and
biologically active substances.

18. A method of using the carrier particles according to claim 13 as carrier particles for culturing biological cells.
